(19) United States
(12) Patent Application Publication
(10) Pub. No.: US 2026/0028657 A1
(43) Pub. Date: Jan. 29, 2026

(54) METHOD OF PURIFYING SOLUTION COMPRISING POLYNUCLEOTIDE

(71) Applicant: DNA Script, Le Kremlin-Bicêtre (FR)

(72) Inventors: Mouaadh KELLAL, Le Kremlin-Bicêtre (FR); Paul BELIN, Le Kremlin-Bicêtre (FR)

(21) Appl. No.: 18/993,240

(22) PCT Filed: Jul. 13, 2023
(86) PCT No.: PCT/EP2023/069431

§ 371 Date: Jan. 10, 2025

(30) Foreign Application Priority Data
Jul. 13, 2022 (FR) FR2207253

(51) Int. Cl. C12P 19/34

(52) U.S. Cl. CPC C12P 19/34

(57) ABSTRACT

The invention relates to a method of purifying a solution comprising newly synthetized polynucleotides, particularly in the field of polynucleotide synthesis such as enzymatic polynucleotide synthesis. The invention also relates to a device configured to carry out the purification method.

### TECHNICAL FIELD

The invention relates to a method of purifying a solution comprising newly synthesized polynucleotides, particularly in the field of polynucleotide synthesis such as enzymatic polynucleotide synthesis. The invention also relates to a device configured to carry out the purification method and a kit thereof.

### INTRODUCTION

Polynucleotide synthesis is increasingly used in the research field and in the pharmaceutical field to provide therapeutic and diagnostic solutions. Examples include genomic and diagnostic sequencing, multiplex nucleic acid amplification, therapeutic antibody development, synthetic biology or nucleic acid amplification.

Polynucleotide synthesis can be done chemically or enzymatically. Either way is classically performed by transferring liquid solutions from filter plates to filter plates which are themselves transferred from stations to stations to isolate the newly synthesized polynucleotides from solid support used during synthesis and possibly from contaminants.

More particularly, polynucleotide synthesis is performed on a solid support in a dedicated first filter plate. Once the synthesis steps are finished, newly synthesized polynucleotide attached to the solid support is transferred from the first filter plate to a second filter plate, also known as transfer plate, to liberate the polynucleotide from the solid support. The liquid solution containing the liberated polynucleotides is finally transferred to a third filter plate called desalting plate to precipitate and purify the polynucleotides from contaminants. Thus, the person skilled in the art commonly uses at least three filter plates and as many transfer steps.

While efficient this method remains costly because of the use of a larger number of filter plates and other consumables which is more costly, less durable and generate more waste.

In addition, the large number of transfer steps, of cleaning steps and of purification steps may result in a lower amount of purified synthesized polynucleotide. Furthermore, it also increases the risk of external contamination. Also, this requires breaking the vacuum many times during the process which lead to a delay and a possible loss of material.

The objective of the present invention is to overcome at least one of the afore-mentioned disadvantages and to furthermore lead to other advantages by proposing a new method for purifying liquid solutions which comprises newly synthesized polynucleotide directly in the first filter plate, eliminating the need to transfer any liquid solution from sample loading to recovery of the newly synthesized polynucleotide. Also, such a method greatly reduces the risk of external contamination in the liquid solution, as well as sample loss and as a more environment friendly approach. Further, the method increases quantity of recovered polynucleotide. The inventors had to overcome the fact that there were many impurities in the plate and that they needed to be sure that were eliminated as they could not do eliminate them during a transfer to another plate. Also, the reagents had to be optimized and selected as the precipitation happen in the same plate and so the ratio of the buffer and the chaotropic agent needs to be optimized.

### SUMMARY OF THE INVENTION

The invention relates to a method of purifying a solution which comprises polynucleotides and contaminants, the solution being contained in a well of a filter plate, the well-being equipped with a filter, the method comprising:
a step of adding a chaotropic agent to the solution contained in the well to get a mixture,
a step of incubating the mixture so that a precipitate and a supernatant are obtained in the well, the precipitate comprising the polynucleotide and the supernatant comprising at least a part of the contaminants,
a step of applying a differential pressure to the well in order to eliminate the supernatant through the filter, the precipitate being retained by the filter,
a step of adding a low salt eluant to dissolve the polynucleotides in the precipitate and transfer the polynucleotides dissolved in the low salt eluant into a container,
wherein said steps of the method is carried out in the well equipped with the filter,
wherein the method is carried out in the well equipped with the filter and the method comprises a step of enzymatically synthesizing the polynucleotide before the step of adding a chaotropic agent, the step of enzymatically synthesizing the polynucleotide being performed in the well in which the step of adding a chaotropic agent is performed.

It should be understood here, and in all that follows, that a filter plate means a plate comprising a plurality of wells, each well being equipped with a filter arranged at a bottom of the well.

The method of purifying polynucleotides contained in a solution along with contaminants proposed by the invention comprises at least four steps that are all performed in the same well of the filter plate. Therefore, there is no need anymore to transfer a solution from one filter plate to another filter plate. By eliminating this transfer the risk of loss of polynucleotide is greatly reduced.

In one or more embodiments, the mixture can be stirred during the second step of incubating. Optionally, stirring is achieved by shaking the filter plate.

The step of enzymatically synthesizing the polynucleotide, the step of adding a chaotropic agent, the step of incubating the mixture, the step of applying a differential pressure and the step of adding a low salt eluant are performed in the same well of a filter plate. Thus, the method of the invention allows to perform the synthesis, the purification and the isolation of the polynucleotides in the same well of a filter plate. It allows to do the purifying directly after the synthesis without any transfer.

In one or more embodiments, the step of enzymatically synthesizing the polynucleotides comprises:
a) a sub-step of providing initiators having a 3′-terminal nucleotide with a free 3′-hydroxyl,
b) a sub-step of contacting under elongation conditions the initiators having free 3′-O-hydroxyls with a 3′-O-blocked nucleoside triphosphate and a polymerase, so that the initiator is elongated by incorporation of a 3′-O-blocked nucleoside triphosphate to form 3′-O-blocked elongated fragments, and
c) a sub-step of deblocking the elongated fragments to form elongated fragments having free 3′-hydroxyls, and
d) a sub-step of repeating sub-steps b) and c), until the polynucleotide is formed,
e) a sub-step of cleaving the polynucleotide from the initiator.

It being understood that each incorporation of one nucleoside triphosphate to extend the initiator constitutes one cycle.

In one or more embodiments, the sub-step of cleaving the polynucleotide from the initiator by enzymatic digestion, by illumination cleavage and/or by chemical cleavage.

In one or more embodiments, the sub-step d) of repeating sub-steps b) and c) is performed by contacting under elongation conditions the elongated fragments obtained in step c), until the polynucleotide is formed.

An example of enzymatic polynucleotide synthesis is described in by e.g. Ybert et al, in International patent publication WO2015/159023.

In one or more embodiments, the method comprises a step of adding beads before the step of incubating the mixture to help to precipitate the mixture. Thus, the beads can be added to the solution before adding the chaotropic agent or the same time. Or the beads can be added to the mixture i.e. after the chaotropic agent has been added. In any event, the chaotropic agent and the beads are added to the same well to get a mixture.

In one or more embodiments, the step of adding beads is preferably performed after the step of synthesis when the method comprises a step of enzymatically synthesizing the polynucleotides.

In one or more embodiments, the beads are glass beads, preferably silica glass beads.

In one or more embodiments, the beads are hollow glass beads, preferably silica hollow glass beads.

In one or more embodiments, a diameter of the beads is comprised between 9 and 13 micrometers.

In one or more embodiments, the filter of the well has pores which have a diameter smaller than a diameter of the beads. Thus, the beads cannot go through the filter of the well. It being understood in the context of the invention that when the method comprises a step of enzymatic synthesis of the polynucleotide before the step of adding a chaotropic agent, at least a first type of bead, preferably an agarose bead, could be used as a support to perform said enzymatic synthesis; and a second type of bead, preferably glass bead, could be used to precipitate the mixture according to the invention, both types of beads being retained by the filter of the well.

In one or more embodiments, the filter has preferably a pore size comprised between 0.45 μm and 1.2 μm.

In one or more embodiments, the filter plate comprises preferably 96 wells or 384 wells.

In one or more embodiments, the chaotropic agent is selected from the group consisting of an isopropanol-based solution, a sodium iodide-based solution, a sodium perchlorate-based solution and one of their combinations. Preferably the chaotropic agent is an isopropanol-based solution.

In one or more embodiments, the step of incubating the mixture to obtain a precipitate is preferably performed at room temperature and preferably for at least 10 min.

In one or more embodiments, a precipitate and a supernatant are formed in the well by incubating the mixture, the supernatant comprising at least a part of the contaminants and the precipitate comprising the polynucleotides and optionally another part of the contaminants.

In one or more embodiments, the method comprises a step of washing the precipitate to eliminate contaminants in the precipitate.

In one or more embodiments, the method comprises:
a step of washing the precipitate with a wash solution to dissolve the contaminants that could be present in the precipitate, and
a step of exerting a differential pressure on the well in order to remove the wash solution through the filter, the wash solution comprising the dissolved contaminants of the precipitate,
   wherein the step of washing the precipitate and the step of exerting a differential pressure are performed after the elimination of the supernatant through the filter, i.e. after the step of applying differential pressure.

In one or more embodiments, the wash solution is an ethanol-based solution, preferably comprising 70% to 80% of ethanol in volume.

In one or more embodiments, the length of the newly synthesized at least one polynucleotide is at least 10 nucleotides long, preferably at least 17 nucleotides long.

The present invention also relates to any kit configured to carry out the method according to any one of the embodiments of the invention, the kit comprising:
chaotropic agent solutions
washing solutions and/or buffer solutions for the purification
eluant solutions and/or buffer to recover the purified polynucleotides
optionally, an instruction manual,
optionally, beads solutions.

In one or more embodiments, the kit is configured to carry out the enzymatic synthesis and the method of the invention, wherein the kit also comprises:
at least one reaction medium containing nucleic acid fragments which are made of nucleotides,
an enzymatic nucleotide addition reagent, nucleotides or combinations of nucleotides suitable for addition by said enzymatic addition reagent,
at least one washing solution and/or at least one buffer solution for the purification phase of the enzymatic synthesis,
at least one amplification reaction medium comprising at least one enzymatic nucleic acid amplification reagent and natural nucleotides suitable for use by the enzymatic amplification reagent.

The invention also relates to a device configured to carry out the method according to any one of the preceding embodiments.

In one or more embodiments, the device comprises a synthesis station and a recovery station, wherein the synthesis station comprises a filter plate located above a pressure adjuster and/or shaker station.

In one or more embodiments, the device can also comprise a cooling or heating station configured to be located below the plate.

Further features and advantages of the invention will become apparent from the following description, on the one hand, and from several embodiments given by way of indication and not limitation with reference to the appended schematic drawings, on the other hand.

### BRIEF DESCRIPTION OF THE DRAWINGS

FIG. 1 represents an embodiment of the invention without the step of washing in the well of the filter plate and comprising a final step of applying differential pressure to the well to recover the purified polynucleotides.

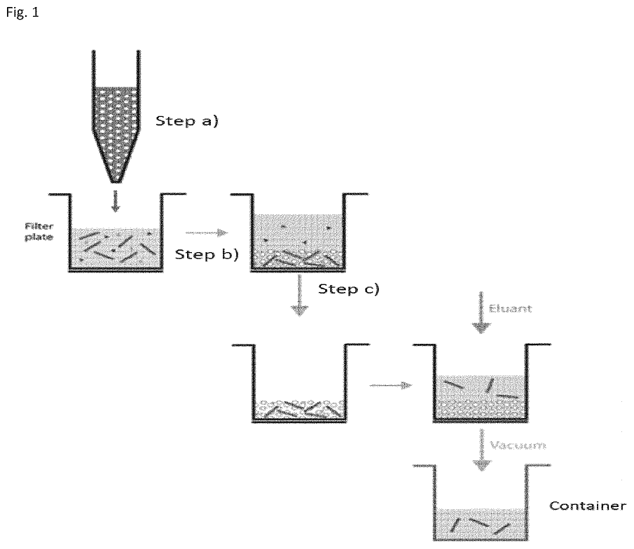

FIG. 2 represents another embodiment of the invention including an additional step of adding a wash solution in the well of the filter plate, and a final step of applying a differential pressure to the well to recover the purified polynucleotides.

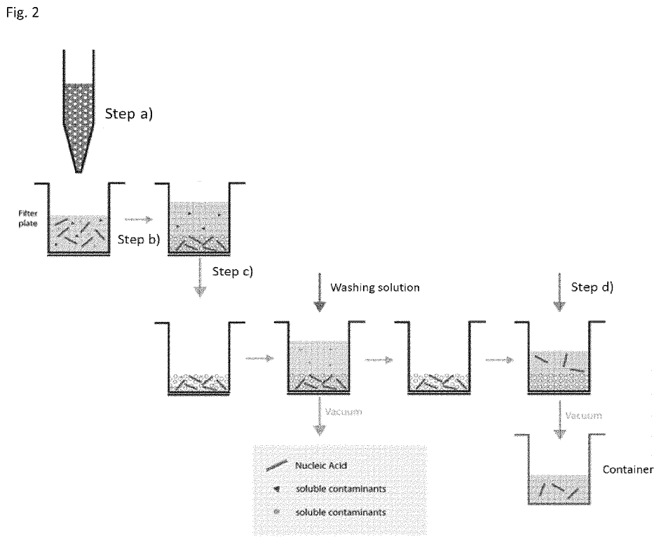

FIG. 3 represents the yield distribution corresponding to the amount of DNA recovered in pmol in each well of the filter plate.

FIG. 4 represents the DNA/salt ratio distribution corresponding to the salt contamination rate in each well of the filter plate.

FIG. 5 represents the DNA/protein ratio distribution corresponding to the protein contamination rate in each well of the filter plate.

FIG. 6A is a gel picture representing the impact of the purity measured by OP2

FIG. 6B represents the purity estimation using % N band of oligos synthetized.

### DETAILED DESCRIPTION OF THE INVENTION

### Definition

“Polynucleotide” is a polymer constituted of nucleotide monomers covalently linked to form a chain or a segment of nucleic acid chains such as DNA or RNA. In the context of the invention, “polynucleotide” and “polynucleotides” can be interchangeable and have the same significance and scope.

“Synthetized polynucleotide” in the context of the invention, means an elongated polynucleotide sequence obtained by enzymatic synthesis process (e.g. WO 2015/159023) until the polynucleotide of interest is obtained.

### Method of the Invention

Classically, the purification of a liquid solution comprising polynucleotides of interest is performed using several stations and plates to extract and finally recover the purified polynucleotide of interest.

However, there in an interest to reduce the number of stations and plates for ecological or economic purposes, but also to improve the automation of high-throughput polynucleotides extraction using basic hardware modules: differential pressure devices such as vacuum, agitation, dispensing and plates. Improving the automation of high-throughput polynucleotide extraction requires the simplification of existing devices in order to extract more and more polynucleotides in a shorter period of time. However, the simplification of existing devices requires first, adapting the purification method and, second, overcoming technical difficulties that have arisen through the adaptation of the prior art methods This is the purpose of the present invention, which aims to limit the transfer of material from one plate to another.

Also, in a first aspect, the invention provides a method of purifying a solution which comprises polynucleotides and contaminants, the solution being contained in a well of a filter plate, the well being equipped with a filter, said method comprising:
a step of adding a chaotropic agent to the solution contained in the well to get a mixture,
a step of incubating the mixture so that a precipitate and a supernatant are obtained in the well, the precipitate comprising the polynucleotide and the supernatant comprising at least a part of the contaminants,
a step of applying a differential pressure to the well in order to eliminate the supernatant through the filter, the precipitate being retained by the filter,
a step of adding a low salt eluant to dissolve the polynucleotides in the precipitate and transfer the polynucleotides dissolved in the low salt eluant into a container,
wherein the method is carried out in the well equipped with the filter.

In particular, the solution is contained in the well of the filter plate, wherein the filter plate is located above a differential pressure station and/or a shaker station.

The filter plate is well known from the person skilled in the art to provide a one-step analysis without requiring centrifugation or precipitation, in particular the filter plate is commonly used to perform PCR analysis in either 96 or 384 well formats.

According to the invention, the method is performed in a single filter plate without the need to transfer the liquid solution comprising the polynucleotides. To do this, the first step requires to add a chaotropic agent in the well of the filter plate to get a mixture. The mixture preferably comprises contaminants, polynucleotides, chaotropic agent and optionally beads.

To improve the precipitation of the polynucleotide of interest and thus purification, the method comprises a second step of incubating the mixture so that a precipitate and a supernatant are obtained in the well, the precipitate comprising the polynucleotides and the supernatant comprising the contaminants.

The chaotropic agent will induce precipitation of the polynucleotide present in the liquid solution, in particular when the liquid solution is in the presence of salt. Chaotropic agent will interfere with the weak intramolecular interactions of the polynucleotide allowing the destruction of its 3-dimensional conformation and consequently its denaturation. Denaturation induces modification of the solubility causing precipitation of the polynucleotide.

According to the invention, all chaotropic agent known from the person skilled in the art could be used. In a preferred embodiment, the chaotropic agent is selected from the group consisting of an isopropanol-based solution, a sodium iodide-based solution, a sodium perchlorate-based solution, and one of their combinations. More preferably the chaotropic agent is an isopropanol-based solution. For example, the volumetric concentration of the isopropanol-based solution is at least 60% (v/v).

In a preferred embodiment, the step of incubating the mixture to obtain a precipitate, is performed at room temperature and more preferably for at least 10 min.

In some embodiments of the invention, the mixture results in the formation of a precipitate comprising polynucleotides and a part of contaminants; and a supernatant comprising another part of contaminants. Thus, the mixture produces a precipitate and a supernatant in the well, the precipitate comprises the polynucleotides and optionally some of the contaminants and the supernatant optionally comprises another part of contaminants. Contaminants could be enzymes, salts, or buffer.

A third step consists of exerting a differential pressure in the well in order to eliminate the supernatant through the filter, and thus the contaminants; the precipitate being retained by the filter. Optionally, the precipitate and the beads being retained together by the filter.

The differential pressure applied to the supernatant does not dissolve the polynucleotides, to eliminate only the contaminants, so the precipitate retained by the filter only contains the polynucleotides of interest and optionally the beads.

The differential pressure applied to the supernatant could be a positive pressure or a vacuum, more preferably a vacuum. For example, the differential pressure can be comprised between 5 and 60 kPa. By “positive pressure”, we mean a pressure higher than atmospheric pressure. The vacuum used is a device commonly used by the person skilled in the art and is configured to be located below the filter plate so that the supernatant can be eliminated from the well through the filter of the filter plate. The precipitate comprising polynucleotide and optionally a part of contaminants is retained by the filters.

In a particular embodiment, when precipitate comprises polynucleotides and a part of contaminants, the method of the invention also comprises a step of washing to dissolves contaminants present in the precipitate.

Thus, in one or more embodiments of the invention, the method also comprises:
a step of washing the precipitate with a wash solution to dissolve the contaminants that could be present in the precipitate, and
a step of exerting a differential pressure on the well in order to remove the wash solution through the filter, the wash solution comprising the dissolved contaminants of the precipitate,
wherein the step of washing and the step of exerting a differential pressure are performed after the elimination of the supernatant through the filter.

In a preferred embodiment, the step of washing and the step of exerting are performed after the elimination of the supernatant through the filter and before the step of adding a low salt eluant.

The wash solution that can be used is any solution that does not dissolve polynucleotide but only the contaminants present in the precipitate, whereby the polynucleotide can then be passed through the wells to remove the contaminants. In a preferred embodiment, the wash solution is an ethanol-based solution, more preferably 70% to 80% ethanol-based solution.

In a preferred embodiment of the invention, the differential pressure applied to the precipitate is also a positive pressure or a vacuum, more preferably a vacuum. The vacuum is also configured to be located below the filter plate so that the contaminants can be eliminated from the well through the filter of the filter plate.

Thus, according to anyone embodiments of the invention, the differential pressure applied to the precipitate and/or the supernatant is a positive pressure or a vacuum, more preferably a vacuum. In particular embodiments, vacuum can be applied to the supernatant and positive pressure to the precipitate or the opposite.

Once the supernatant is eliminated from the well, a fourth step of adding a low salt eluant aims to solubilize the precipitate retained by the filter, in particular to re-suspend and dissolve the polynucleotides. Dissolution is particularly useful in order to transfer them into the desired container. By “low salt eluant”, we mean eluant without the majority of mineral salts present in water, preferably the low salt eluant is purified water, or distilled water, more preferably ultra-pure water adapted for PCR as known from the person skilled in the art, e.g. Molecular Biology Grade Water (Nuclease & Protease free).

Thus, the dry polynucleotides obtained after eliminating the supernatant, can then be recovered using a low salt eluant solution into the desired container.

The desired container is preferably a well of a recovery plate.

In another preferred embodiment, the low salt eluant is selected from the group consisting in purified water, distilled water, ultra-pure water, and one of their combinations.

According to the invention, the length of the newly synthetized at least one polynucleotide is at least 10 nucleotides long, preferably at least 17 nucleotides long. In a more preferred embodiment according to the invention, the method comprises:
A step of adding a chaotropic agent to the solution contained in the well to get a mixture,
A step of incubating the mixture so that a precipitate and a supernatant are obtained in the well, the precipitate comprising the polynucleotides and optionally contaminants and the supernatant comprising at least a part of the contaminants,
A step of applying a differential pressure to the well in order to eliminate the supernatant through the filter, the precipitate being retained by the filter,
A step of washing the precipitate with a wash solution to dissolve the contaminants that could be present in the precipitate, and
A step of exerting a differential pressure on the well in order to remove the wash solution through the filter, the wash solution comprising the dissolved contaminants of the precipitate,
A step of adding a low salt eluant to dissolve the polynucleotides in the precipitate and transfer the polynucleotides dissolved in the low salt eluant into the desired container,
wherein each step of the method is carried out in the well equipped with the filter.

In one or more embodiments of the invention, the method comprises a further step of adding beads before the step of incubating the mixture. The addition of beads simultaneously with the chaotropic agent in the well to get a mixture is particularly suitable to act as a precipitating surface and promote polynucleotide precipitation. The precipitation is further improved when the beads are glass beads, preferably silica glass beads.

In a more preferred embodiment, the beads are glass beads, which are hollow beads. It is particularly suitable that the glass spheres are hollow to have a density equivalent or slightly higher than the chaotropic agent to allow easy resuspension.

The diameter of the beads is preferably comprised between 9 and 13 micrometers. When beads are added, the method of the invention is performed in a well of a filter plate, wherein the filter has pores which have a diameter smaller than a diameter of the beads. Preferably, the filter has a pore size comprised between 0.45 μm and 1.2 μm.

Once the supernatant and precipitate comprising beads have been formed, the wells are flushed. Due to the particular diameter of the beads, the precipitate comprising the beads and polynucleotides is thus retained by the filters of the filter plate.

In a preferred embodiment, the method of the invention is performed in the context of enzymatic synthesis. Thus, the method preferably comprises a further step of enzymatically synthesizing the polynucleotide before the step of adding a chaotropic agent, the step of enzymatically synthesizing the polynucleotide being performed in the well in which the step of adding a chaotropic agent is performed. Thereby, the synthesis of the polynucleotide is also performed in the well in which the purification method according to the present invention is performed.

Preferably, the further steps of enzymatically synthesizing the polynucleotide comprises:
a) A sub-step of providing initiators having a 3′-terminal nucleotide with a free 3′-hydroxyl,
b) A sub-step of contacting under elongation conditions the initiator having free 3′-O-hydroxyls with a 3′-O-blocked nucleoside triphosphate and a polymerase, so that the initiator is elongated by incorporation of a 3′-O-blocked nucleoside triphosphate to form 3′-O-blocked elongated fragments, and
c) A sub-step of deblocking the elongated fragments to form elongated fragments having free 3′-hydroxyls, and
d) A sub-step of repeating substeps b) and c), until the polynucleotide is formed,
e) A sub-step of cleaving the polynucleotide from the initiator, preferably by endonuclease digestion.

In a preferred embodiment, the substep d) of repeating substeps b) and b) is performed by contacting under elongation conditions the elongated fragments obtained in step c), until the polynucleotide is obtained.

A very large number of DNA polymerases exists, which are capable of catalyzing the synthesis of a polynucleotide in the presence or absence of a template strand. Thus, the DNA polymerases of the polX family are involved in a large range of biological processes, in particular in DNA repair mechanisms or mechanisms for the correction of errors appearing in DNA sequences. These enzymes are capable of inserting nucleotides, which have undergone excisions after the identification of sequence errors, in the nucleic acid strands. The DNA polymerases of the polX family comprise the DNA polymerases β (Pol β), λ (Pol λ), μ (Pol μ), yeast IV (Pol IV), and the terminal deoxyribonucleotidyl transferase (TdT). TdT in particular is particularly useful and thus, used very widely in the methods of enzymatic synthesis of nucleic acid molecules.

However, these DNA polymerases allow only the incorporation of natural nucleotides. In all cases, the natural DNA polymerases lose their catalytic activity in the presence of non-natural nucleotides and in particular 3′-OH modified nucleotides which exhibit greater steric hindrance than the natural nucleotides. Therefore, enzymes that are capable of catalyzing the synthesis of a polynucleotide by incorporating such nucleotides has been developed. In particular, DNA polymerase variants, preferably TdT variant have been developed, for example as described in US 2020/0002690.

In more preferred embodiment, the enzymatic synthesis of the polynucleotide is described in by e.g. Ybert et al, in International patent publication WO2015/159023.

### Kit of the Invention

The present invention also relates to any kit configured to carry out the method according to any one of the preceding embodiments, comprising:
chaotropic agent solutions
washing solutions and/or buffer solutions for the purification
eluant solutions and/or buffer to recover the purified polynucleotides,
optionally, an instruction manual,
optionally, beads solutions.

In a preferred embodiment, the kit is configured to carry out the enzymatic synthesis and the method of the invention, wherein the kit also comprises:
At least one reaction medium containing nucleic acid fragments comprising n nucleotides
an enzymatic nucleotide addition reagent, nucleotides or combinations of nucleotides suitable for addition by said enzymatic addition reagent,
at least one washing solutions and/or buffer for the purification phase of the enzymatic synthesis,
at least one amplification reaction medium comprising at least one enzymatic nucleic acid amplification reagent, and natural nucleotides suitable for use by the enzymatic amplification reagent.

Different types of kits can be offered according to the needs of the experimenter. Similarly, different types of kits can be offered depending on whether or not they are to be used automatically.

### Device of the Invention

In another aspect, the invention also relates to a device configured to carry out the method according to any one of the preceding embodiments.

In a preferred embodiment, the device comprises a synthesis station and a recovery station, wherein the synthesis station comprises a filter plate (11) located above a pressure adjuster and/or shaker station.

Preferably, the device also comprises a cooling or heating station configured to be located below the plate. The cooling or heating station is particularly useful to specifically control the temperature of the wells and consequently of the solution contained in the wells in order to e.g. heat to release the synthesized polynucleotides coupled to the solid support used for the enzymatic synthesis or cool rapidly to room temperature to perform the method according to the invention.

Other features and advantages of the invention will be more clear from the following examples and results which are of course non-limiting.

### EXAMPLES

The example demonstrates the efficiency of the method of the invention, in particular by carrying out an enzymatic synthesis followed by the synthesis and purification method of the invention in the same filter plate.

### Materiel & Method

750 pmol of an initiator DNA strand, which is a small DNA strand, having a 3′-terminal nucleotide having a free 3′-hydroxyl, used to initiate the enzymatic synthesis and elongated the DNA strand of interest, as described in the international patent application WO 2015/159023. The initiator is loaded on solid support and then distributed to each well of a 384 w filter-plate. The filter pores are 1.2 μm wide and the membrane is made of polyethersulfone (PES).

The loaded plate is placed in the device marketed by DNA Script under the trade name SYNTAX® to perform the enzymatic synthesis method described in WO 2015/159023. The enzymatic synthesis is carried out on a set of custom sequences. (16 DNA probes from 20 to 28 mers)

After the synthesis, the following steps, described in Table 1 hereinafter, are performed in each well of the synthesis plate.

The first step reads as follows: “25 μL of low salt eluant (MB water) is dispensed in every well of the plate, the plate is then incubated at Room Temperature and 1500 rpm for 5 s, liquid is then evacuated during 30 s at −40 kPa, this step is repeated twice”.

TABLE 1

| | | | | Inc | Shake | | Evacuation | |
|---|---|---|---|---|---|---|---|---|
| | | | Volume | Time | speed | Temp | Pressure | Evacuation |
| Step | Buffer | Number | (uL) | (s) | (rpm) | (° C.) | delta (kPa) | Time (s) |
| PREWASH | SE | 2 | 25 | 5 | 1500 | 46 | 40 | 30 |
| PREWASH | W3 | 2 | 50 | 300 | 1000 | 46 | 40 | 30 |
| PREWASH | SE | 2 | 25 | 5 | 1500 | 46 | 40 | 30 |
| PREWASH | W1 | 3 | 25 | 30 | 1500 | 46 | 40 | 30 |
| DRYING | — | — | — | — | — | 75 | 40 | 1200 |
| LIBERATION | LB | 1 | 27 | 1800 | 1500 | 65 | — | — |
| PRECIPITATION | W6 | 1 | 53 | 1800 | 0 | RT | 40 | 60 |
| DESALTING | W1 | 2 | 70 | 10 | 0 | RT | 40 | 60 |
| DRYING | — | — | — | — | — | RT | 40 | 1200 |
| ELUTION | SE | 2 | 25 | 180 | 1500 | RT | 40 | 60 |

SE buffer us law salt eluant (MB water). W3 buffer is 10 mM Tris, 0.25 mM EDTA, 100 mM NaCl, 0.5% Tween, Proteinase K 0.1 mg/mL. LB buffer is 10 mM Tris, 500 mM MgCl2, 1700 mM NaCl, Endo V 2.9 μM. W1 buffer is 80% EtOH 20% Mili Q Water. W6 buffer is Pure isopropanol.

To demonstrate the efficacy of the method, success criteria have been determined:
C1: The process is 100% automated in a SYNTAX® device using a single filter plate for the synthesis and the method according to the invention,
C2: DNA recovery must achieve a Yield >200 pmol for 100% of the wells (for 750 pmol loaded, measured with 260 nm absorbance),
C3: Salt contamination must be:

$$\frac{A 260 nm}{A 230 nm} > 1.8 for 100 \% of the wells ,$$

C4: Protein contamination must be:

$$\frac{A 260 nm}{A 280 nm} > 1.6 for 100 \% of the wells ,$$

C5: The method according to the invention should have no impact on the purity of synthetized strands.

The samples are then recovered in a 384 UV quantification plate and analyzed. The inventors analyzed the “Yield” of DNA recovered, salt or protein contamination and checked for purity.

The amount of DNA recovered in pmol is referred to as “Yield”, typically the quantity of initiator DNA loaded is 750 pmol which sets the maximum recovery yield at 750 pmol. In practice we recover from 200 to 400 pmol of DNA depending on protocol used, solid support, etc. . . . .

DNA is then recovered in a UV quantification plate and quantified in an Epoch microplate spectrophotometer using 260 nm absorbance. The inventors also use 230 nm and 280 nm to estimate respectively salt and protein contamination. The 260/230 values for “pure” nucleic acid are higher than the respective 260/280 values.

Expected 260/230 values are commonly in the range of 2.0-2.2. If the ratio is significantly lower than expected, it may indicate the presence of contaminants which absorb at 230 nm.

Expected 260/280 values are commonly in the range 1.8-2.0. If the ratio is significantly lower than expected, it may indicate the presence of contaminants which absorb at 280 nm.

The DNA synthetized is then analyzed using the Oligo Pro II (OP2) system, it utilizes UV detection along with parallel capillary electrophoresis to provide single nucleotide resolution and direct assessment of oligonucleotide purity.

### Results

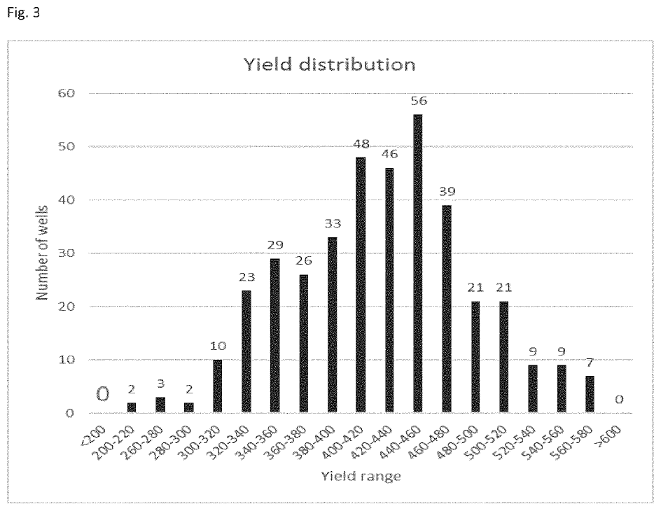

Results are presented in FIG. 3 to FIG. 6B.

According to the first criterion C1, the present method has been automated with minimal hardware and software requirements using a single filter plate, without human intervention.

Prior to the purification method according to the invention, 750 pmol of DNA is loaded into each well of the plate. As shown in FIG. 3, no well comprises less than 200 pmol. Furthermore, the inventors have demonstrated that more than 50% of the wells comprise at least 400 pmol of DNA demonstrating a very good efficiency of the process according to the invention, in the order of at least 10-20% efficiency compared to the results obtained by the methods of the prior art.

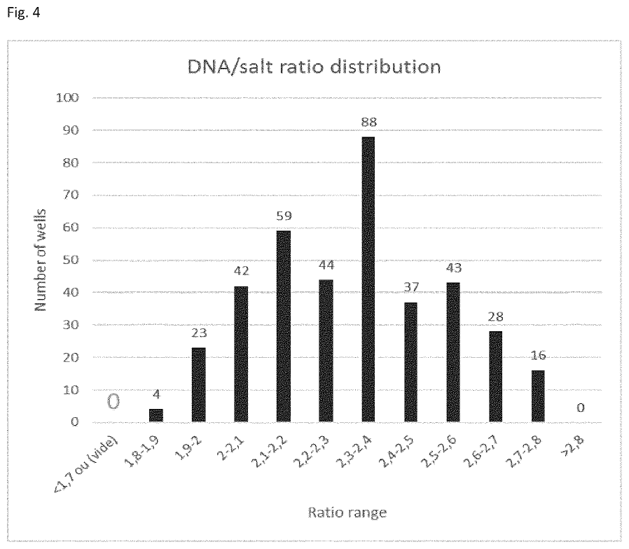

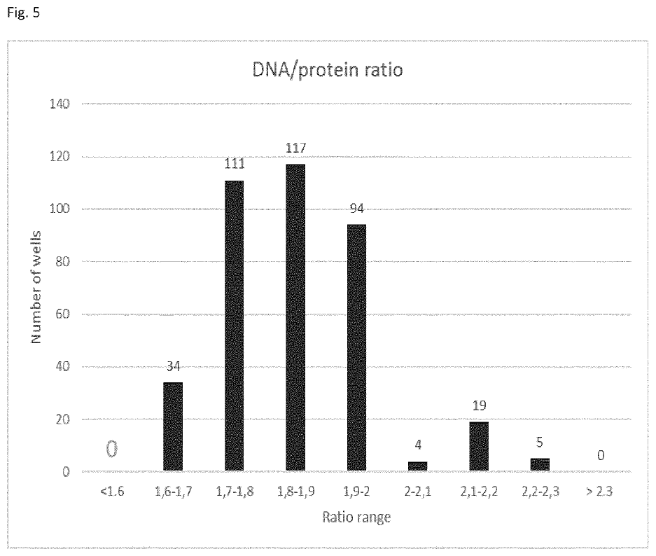

The inventors have also shown that the contamination in each well is very low. To check the contamination, the inventors measured the DNA/salt ratio and then the DNA/protein ratio. Thus, the inventors showed that the DNA/salt ratio is at least 1.8 for all wells in FIG. 4 and the DNA/protein ratio is at least 1.6 for all wells in FIG. 5. These results also represent an increase in recovered polynucleotides of around 20% compared to the prior art.

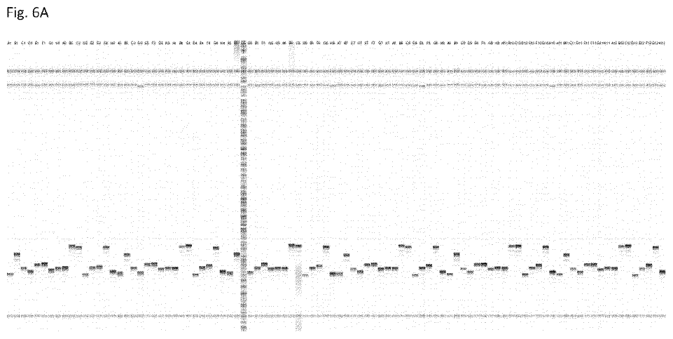

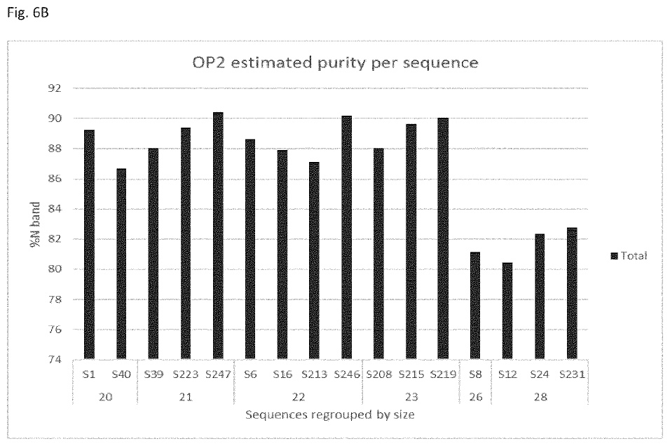

Finally, the inventors have demonstrated that the method of the invention has no impact on the purity of the synthesized strands, for which the oligos were analyzed using OP2. The results are shown in FIG. 6A and FIG. 6B, showing that the purity of the oligos corresponds to the best standard for this given chemistry and oligo size.

To conclude, the method of the invention meets all requirements of a performant purification method. This opens new perspectives for devices incorporating such a process, as well as new functionalities.

1. A method of purifying a solution which comprises polynucleotides and contaminants, the solution being contained in a well of a filter plate, the well being equipped with a filter, said method comprising:
(i) a step of adding a chaotropic agent to the solution contained in the well to get a mixture,
(ii) a step of incubating the mixture so that a precipitate and a supernatant are obtained in the well, the precipitate comprising the polynucleotide and the supernatant comprising at least a part of the contaminants,
(iii) a step of applying a differential pressure to the well in order to eliminate the supernatant through the filter, the precipitate being retained by the filter, and
(iv) a step of adding a low salt eluant to dissolve the polynucleotides in the precipitate and transfer the polynucleotides dissolved in the low salt eluant into a container,
wherein the method is carried out in the well equipped with the filter and the method comprises a step of enzymatically synthesizing the polynucleotide before the step of adding a chaotropic agent, the step of enzymatically synthesizing the polynucleotide being performed in the well in which the step of adding a chaotropic agent is performed.

2. The method of claim 1, wherein the step of enzymatically synthesizing the polynucleotide comprises:
a) A sub-step of providing initiators having a 3′-terminal nucleotide with a free 3′-hydroxyl,
b) A sub-step of contacting under elongation conditions the initiators having free 3′-O-hydroxyls with a 3′-O-blocked nucleoside triphosphate and a polymerase, so that the initiator is elongated by incorporation of a 3′-O-blocked nucleoside triphosphate to form 3′-O-blocked elongated fragments, and
c) A sub-step of deblocking the elongated fragments to form elongated fragments having free 3′-hydroxyls, and
d) A sub-step of repeating substeps b) and c) until the polynucleotide is formed, and
e) A sub-step of cleaving the polynucleotide from the initiator.

3. The method of claim 2, wherein the sub-step of cleaving the polynucleotide from the initiator by enzymatic digestion, by illumination cleavage and/or by chemical cleavage.

4. The method of claim 1, wherein step of incubating is performed at room temperature.

5. The method of claim 1, wherein step of incubating is performed for at least 10 min

6. The method of claim 1, wherein the filter plate comprises preferably 96 wells or 384 wells

7. The method of claim 1, wherein the chaotropic agent is selected from the group consisting of an isopropanol-based solution, a sodium iodide-based solution, a sodium perchlorate-based solution, and one of their combinations.

8. The method of claim 1, wherein a precipitate and a supernatant are formed in the well by incubating the mixture, the supernatant comprising at least a part of the contaminants and the precipitate comprising the polynucleotides and optionally another part of the contaminants.

9. The method of claim 1, wherein the method comprises:
A step of washing the precipitate with a wash solution to dissolve contaminants that could be present in the precipitate, and
A step of exerting a differential pressure on the well in order to remove the wash solution through the filter, the wash solution comprising the dissolved contaminants of the precipitate,
wherein the step of washing the precipitate and the step of exerting a differential pressure are performed after the elimination of the supernatant through the filter.

10. The method of claim 1, wherein the wash solution is an ethanol-based solution, preferably comprising 70% to 80% of ethanol in volume.

11. The method of claim 1, wherein the method comprises a step of adding beads before the step of incubating the mixture.

12. The method of claim 1, wherein the filter of the well has pores which have a diameter smaller than a diameter of the beads.

13. The method of claim 11, wherein the beads are glass beads, preferably silica glass beads.

14. The method of claim 1, wherein the beads are hollow beads.

15. The method of claim 1, wherein the filter has a pore size comprised between 0.45 μm and 1.2 μm.

16. The method of claim 11, wherein a diameter of the beads is comprised between 9 and 13 micrometers.

17. The method of claim 1, wherein the length of the newly synthetized at least one polynucleotide is at least 10 nucleotides long, preferably at least 17 nucleotides long.

18. A kit configured to carry out the method of claim 1, comprising:
(i) chaotropic agent solutions;
(ii) washing solutions and/or buffer solutions for the purification; and
(iii) eluant solutions and/or buffer to recover the purified polynucleotides.

19. The kit according to claim 18, wherein the kit also comprises:
(iv) At least one reaction medium containing nucleic acid fragments comprising n nucleotides
(v) an enzymatic nucleotide addition reagent, nucleotides or combinations of nucleotides suitable for addition by said enzymatic addition reagent,
(vi) at least one washing solutions and/or buffer for the purification phase of the enzymatic synthesis,
(vii) at least one amplification reaction medium comprising at least one enzymatic nucleic acid amplification reagent, and natural nucleotides suitable for use by the enzymatic amplification reagent.

20. A device configured to carry out the method of claim 1.

21-22. (canceled)
